(19) United States
(12) Patent Application Publication
(10) Pub. No.: US 2008/0262251 A1
(43) Pub. Date: Oct. 23, 2008

(54) Process For the Recovery of Sterols From Organic Material

(76) Inventors: Setsuo Sato, Sao Jose dos Campos (BR); Wolfgang Albiez, Illertissen (DE); Alexssander S. Araujo, Sao Jose dos Campos (BR); Wanderson Bueno De Almeida, Sao Jose dos Campos (BR)

Correspondence Address: SYNNESTVEDT & LECHNER LLP, 1101 MARKET STREET, PHILADELPHIA, PA, 19107, US

(21) Appl. No.: 11/596,384

(22) PCT Filed: May 31, 2004
(86) PCT No.: PCT/BR2004/000082

§ 371 Date: Nov. 14, 2006

(51) Int. Cl. C07J 9/00

(52) U.S. Cl. 552/545

(57) ABSTRACT

A process for recovering sterols from organic material containing sterols and sterol derivatives. In a first step (a) the organic material is reacted with at least one of polyols, polyamines, alkanolamines or monohydric alcohols to increase the amount of free sterols in the organic material; (b) reacting residual reactants, and their esters or amides with epoxydated components; and (c) separating the free sterols from the mixture by short path distillation, thin film evaporation, or flash evaporation. The recovered sterols can be further purified by a crystallization step.

### OBJECT OF THE INVENTION

The present invention generally belongs to the area of chemical processes of isolating and purifying constituents from natural sources and, in particular, it relates to a new method for separating concentrating and purifying sterols from organic material.

### STATE OF THE ART

Numerous methods have been described for the recovery of sterols from organic material converting sterol fatty esters into free sterols with sub-sequent purification by distillation and crystallization.

The separation of sterols by transesterification and saponification steps followed by further esterification or solvent extraction often requires organic solvents, results to large amounts of salts as waste and usually needs many process steps, so that the process is less economic and less environmentally friendly and results in relatively low yields.

The U.S. Pat. No. 6,344,573 B1 relates to a process for the extraction and concentration of unsaponifiables substances from residues of animal or vegetable products. This process does not require organic solvents but involves several process steps creating a high amount of by-products.

The European patent application EP 1291355 A1 discloses a process for recovering sterols and/or wax alcohols from a crude tall oil material containing sterols and/or wax alcohols in esterified form and fatty and/or rosin acids and optionally sterols and/or wax alcohols in free form. Said method is comprising the steps of: converting free acids in the source material to corresponding salts, removing water if present, transesterifying the esterified sterols and/or wax alcohols present in the dry material obtained in step a or step b to liberate sterols and/or wax alcohols, evaporative fractionating the trans-esterified material, and isolating sterols and/or wax alcohols from the obtained fractions or the residue.

After liberating the bonded sterols, the material is submitted to a fractionation step to separate the sterols from the other components. Sterols remain in the residue stream and light end components are distilled off. This process consists of a large number of different steps bearing the risk to decrease the final yield of free sterols.

A high efficiency continuous process for recovering high purity sterol mixtures from organic material including tall oil pitch is described in the European patent application EP 1081156 A2 comprising the steps of distillation, crystallization and recirculation of the mother liquor residue. In order to achieve the high purity many production steps and the use of organic solvents was necessary.

The International application WO 00/64921 discloses a process to purify sterols from natural sources by complexation with a metal salt, this process has less process steps, but requires a large amount of solvents.

Sterol concentrate purification, such as the evaporative fractionation disclosed in the European patent application EP 1 389 622 A2 does avoid salts and organic impurities, but again includes many process steps. The European patent EP 0 260 243 B1 describes a method to set free sterol from organic materials by treating said material with ammonia or amines in order to avoid the addition of inorganic salts which complicates further purification steps.

It has been an object of the present invention to provide an efficient economical, environmentally friendly process which provides a high process yield in the recovery of sterols and a high final sterol purity.

### DETAILED DESCRIPTION OF THE INVENTION

The present invention provides a process for the recovery of sterols from organic material, which comprises

a) reacting organic material containing free sterols and sterol derivatives with

(i) polyols or

(ii) polyamines or

(iii) alkanolamines or

(iv) alcohols

to convert the sterol derivatives into free sterols
b) reacting the residual reactants and their esters or amides with epoxydated substances and subsequently
c) separating the free sterols from the remaining components by distillation.

As used herein the term “sterols” refers to any plant or animal based sterol or sterol derivative for example cholesterol, sitosterol, campesterol, ergosterol, stigmasterol, brassicasterol, avenasterol as well as the saturated components called stanols like sitostanol, stigmastanol and campestanol and their derivatives.

In nature sterols can occur as free sterols or predominantly derivatized as sterol esters of long-chain fatty acids. During processing which is usually conducted at higher temperatures, low pressure, and relatively long residence time, most of the sterols in organic material are further derivatized.

Since sterol derivatives are difficult to separate or even purify either via distillation, extraction or direct crystallization it is necessary to convert them back to free sterols before proceeding with further processing steps. This high number of process steps renders a process uneconomical, so that one object of the current invention is the reduction of process steps. The enrichment of free sterols from natural organic material is achieved by step a) of the proposed process. Bonded sterols are converted back into free sterols using an appropriate reactant which combines a high transesterification or transamidation reaction yield with the conversion of fatty and other organic acids into their respective mono-, di- and polyesters, aminoamides, diamides, and polyaminoamides. Some reactants do neither require a catalyst, reactant excess nor high pressure to achieve complete transesterification or transamidation, in addition no solvent is used.

In order to prevent that the low molecular weight products formed or reactants left after transesterification or transamidation such as long chain alcohols and polyalcohols, glycols and polyglycols, amines and polyamines, alkanolamines, monoesters of polyalcohols, or amidoamines will distill off together with the free sterols and contaminate the final product, the molecular weight of the non-free-sterol-material is increased. The crucial step of the process is therefore the reaction of the residual reactants and their respective alkanol esters or alkanolaminoamides left after step a) with epoxydated substances. The higher molecular weight of the residuants after reacting with the epoxyde groups results in a decreased vapor pressure, so that distillative separation of free sterols from the residuants is improved. This will avoid contamination with light end materials and increase the sterol concentration in the distillate. Free sterols can now easily be separated using a distillation apparatus with almost no theoretical separation stages such as molecular distillation, short path distillation or thin film evaporators. This process provides a distillate with a sufficient sterol concentration to produce sterols with a purity of more than 98% using only one single crystallization step.

Less reaction steps and a single distillation reduce processing costs significantly.

The organic material (feed) used in step a) can be any material containing free sterols or sterol esters or other sterol derivatives, such as vegetable oil distillates (VOD) and/or deodorizer distillates (DOD), residues of fatty acids (FAR) and/or fatty acid methyl or ethyl ester production (FAMER, FAEER), soap stock fatty acid residues (SSFAR), sugar cane wax (SCW), crude tall oil (CTO) and tall oil pitch (TOP). Due to its availability and amount of sterols or sterol derivatives the preferred feed is tall oil pitch.

Deodorizer distillate (DOD) or Vegetable Oil Distillate (VOD) from the deodorization process of vegetable oils, usually contain 2-15 wt % of sterols, using this type of feed concentrations of up to 45 wt % of free sterols based on the weight of the concentrate can be produced.

Residues from distillation of either fatty acids (FAR) or methyl esters (FAMER) contain 2-30 wt % of sterols and can also be concentrated up to 45 wt % free sterols based on the weight of the concentrate by the current technique, as well as crude tall oil (CTO) from the sulphatation employed in cellulose manufacturing, which contains 3-7 wt % sterol or tall oil pitch (TOP) derived from the process of paper production from wood which usually has 8-16 wt % sterols. Even other organic materials such as sugar cane waxes (SCW) and soap stock fatty acids (SSFA) with a sterol concentration below 5 wt % can be concentrated up to 45 wt % sterols based on the weight of the concentrate using the said process. Surprisingly it was found that the inventive process is always resulting in a high concentration of free sterols even if the organic material of step a) contains lower amounts of sterols. It is an important advantage that the yield of sterols from this process is independent from the organic material resp. the sterol concentration of the feed.

In step a) of the inventive process this organic material is reacted with long chain alcohols or polyalcohols, glycols or polyglycols, amines or polyamines, or alkanolamines and sterol esters and other sterol derivatives are transformed into the free sterols while other reactants result in the correspondent derivatives such as alkanol esters or amides.

Examples for polyols that could be used are
(i) polyols such as ethylene, diethylene glycol, triethylene, tetraethylene, and polyethylene glycols with molecular weights up to 1000, propylene glycol and polypropylene glycol having molecular weights up to 1000, glycerine and polyglycerines, mono and di-pentaerythritol, trimethylol-propane, high molecular weight polyols such as C7 to C24 fatty alcohols.
   Suitable polyamines are
(ii) ethylenediamine, triethylenetetramine, tetraethyleneamine or dimethylpropilamines.
   Alkanolamines for step a) could be
(iii) monoethanolamine, diethanolamine, triethalonamine, aminoethylethanolamine, dimethylaminopropylamine.
   The alcohols for this process step are
(iv) either from an Oxo process or linear alcohols from natural sources including the Guebert alcohols with C6 to C36 carbon atoms.

The preferred reactants are polyamines, most preferred is ethylendiamine. The use of ethylendiamine enables the reaction without the use of a catalyst, avoiding solvents and high pressure.

The amount of the reactants has to be adapted concerning the saponification value of the used feed. The molar ratio of reactant ((i) to (iv)) in the transesterification and/or transamidation step (a) in relation to the saponification number of the organic material (feed) is 0.5 to 6.

The transesterification or transamidation is run in the presence of metallic catalysts, preferred are zinc oxide, sodium-, potassium- or lithium hydroxide and organic tin catalysts. Some reactants—such as alkanolamines, especially diethanolamine, do not require the use of a catalyst.

Step a) and b) can be conducted in form of a batch process (examples 1-5) or in form of a continuous reaction process (examples 6, 7).

The batch process is conducted at temperatures of 150 to 290° C., preferably 180 to 270° C., the best yield and purity is achieved at temperatures of 210 to 260° C.

The continuous reaction process starts at ambient temperature of 20 to 30° C. and the temperature is increased up to 240 to 330° C., preferably 250 to 310° C. and most preferred up to 270 to 300° C., while the pressure increases up to 1 to 25 bar, preferably 3 to 10 bar.

Step a) is followed by a quick reaction of the residual reactants and their respective alkanol esters or alkanolaminoamides left in the reaction product with the epoxy groups of the epoxydated substances.

Generally all materials with epoxy groups can be used for this reaction, preferred are epoxydated vegetable oils such as epoxydated soy bean oil (ESO), epoxydated linseed oil (ELO), epoxydated sunflower oil or epoxydated lard oil or epoxydated fatty acids and epoxydated fatty acid esters such as tall oil fatty acids, oleic and linoleic acids. Most preferred are epoxydated soy bean oil (ESO) and/or epoxydated linseed oil (ELO).

In order to achieve the best results an amount of 2 to 20 wt % epoxydated vegetable oils preferably 5 to 10 wt % based on the amount of the feed used in step a) is added.

The temperature for the reaction of step b) is 120 to 220° C., preferably 150° to 200° C., especially 170° C. to 190° C. if the process is conducted as a batch process. In case of a continuous process the high temperature during step a) is rapidly cooled down after addition of the epoxydated material.

Without addition of the epoxydated substances the overall yield of free sterols is only 30 wt % while with step b) a free sterol yield of 40 to 45 wt % based on the weight of the concentrate can be achieved.

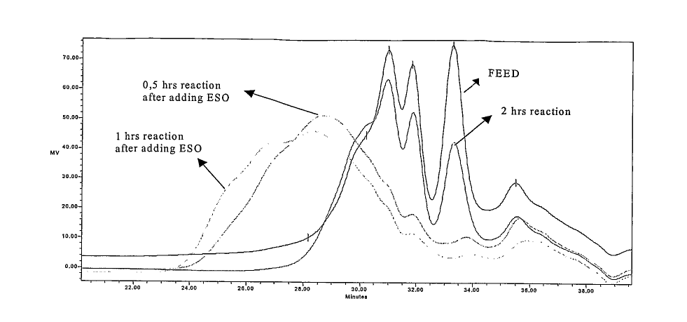

The result of a Gel Permeation Chromatography (GPC) in FIG. 1 shows the process principle and the typical molecular weight growing profile from a typical feed during the transesterification or transamidation process, and after adding the epoxydated material.

(Line 1 represents the organic material (feed), line 2 the reaction product after 2 h reaction, line 3 is the reaction product 0.5 h after addition of ESO and line 4 represents the reaction product 1 h after addition of ESO).

Free sterols can then easily be separated using single distillation apparatus such as short path or thin film evaporation equipment, flash evaporators or molecular distillation. The possibility to use preferably short path distillation or molecular distillation enables the use of low distillation temperatures and low vacuum to avoid degradation and loss of sterols. The high reaction yield after step b) combined with a short path distillation results in a very short residence time avoiding sterols degradation or any other side reactions such as sterol ester formation and results in a process yield of more than 95% based on the total amount of sterols.

Most preferred is a short path destillation, which is operated at a reduced pressure of 0.01 to 10 mm/Hg and a temperature of 180 to 310° C. and/or a fractionation column which is operated at 0.1 to 5 mmHg and a temperature of 170 to 230° C. at the top and 240 to 280° C. at the bottom.

After concentration of the sterols a final crystallisation process can be added as step d) in order to purify the sterols. Preferably this crystallisation does just include one crystallisation cycle. A sterol purification of 98 wt % and higher can be reached in just one single crystallisation step using the concentrated sterols resulting from step a to c).

### EXAMPLES

The examples describe the use of polyalcohols, long chain alcohols, polyamines and alkanolamines as reactants to set the sterols free by trans-esterification or transamidation of vegetable oil distillate (VOD), crude tall oil (CTO), tall oil pitch (TOP), fatty acid methyl ester residues (FAMER), sugar cane wax (SCW). After releasing the sterols and reaction of the residual reactants and their esters or amides with epoxydated products this material is distilled at high temperature and low pressure in order to separate the free sterols from the other high boiling point components.

Step a)—Transesterification and/or Transamidation Step and Step b)

### Example 1

1 kg of TOP were reacted with 155 g (2.53 moles) monoethanolamine from Aldrich Chemical Co. and 10 g zinc oxide (0.12 moles) from Merck KGaA, in a 2-liter, 3-necked round bottom flask equipped with a thermometer and mechanical agitator at a temperature of 220° C. for 5 h. Then 50 g of epoxydated soy bean oil (ESO) were added to the flask, reaction temperature was maintained at 180° C. and the reaction was continued for 1 hour. The yield of sterol release—relation of free sterols in the reaction product after step a and b compared to the total amount of free and derivatized sterols in the feed—was 99.34%.

### Example 2

1 kg of VOD was treated with 326 g (3.1 moles) of diethyleneglycol from Aldrich Chemical Co. and 17 g of zinc oxide (0.21 moles) from Merck KGaA, in a 2-liter, 3-necked round bottom flask equipped with a thermometer and mechanical agitator at a temperature of 220° C. After 5 hours 80 g of ESO were added to the flask, reaction temperature was maintained at 180° C. and the reaction was continued for 1 hour. The yield of sterol release was 90.74%.

### Example 3

1 kg of TOP was reacted with 60 g (1.00 moles) of ethylenediamine from Aldrich Chemical Co. into a 2-liter, 3-necked round bottom flask equipped with a thermometer and mechanical agitator at a temperature of 220° C. for 3 h. After reaction completion 70 g of ESO were added to the flask, reaction temperature was maintained at 180° C. and the reaction was continued for 1 hour. The yield of sterol liberation was 99.7%.

### Example 4

1 kg of FAMER was reacted with 470 g (5.11 moles) of Glycerol from Aldrich Chemical Co. and 1.7 g of Lithium Hydroxide (1.0 mol) from Merck KGaA, in a 2-liter, 3-necked round bottom flask equipped with a thermometer and mechanical agitator at a temperature of 240° C. After 5 h 60 g of ESO were added to the flask, reaction temperature was maintained at 180° C. and the reaction was continued for 1 hour. The yield of sterol liberation was 85%.

### Example 5

1 kg of VOD was reacted with 90 g of ethylenediamine (1.5 moles) obtained from Aldrich Chemical Co., in a 2-liter, 3-necked round bottom flask equipped with a thermometer and mechanical agitator at a temperature of 220° C. After 4 hours 90 g of ESO were added to the flask, reaction temperature was maintained at 180° C. and the reaction was continued for 1 hour. The yield of sterol release was 99.5%.

### Example 6

0.9 kg of TOP were mixed with 60 g of ethylenediamine (1.0 moles) obtained from Aldrich Chemical Co., and transferred into a pressure reactor. Within 20 minutes temperature was increased from initially 25° C. to 290° C. and maintained for another 20 minutes, a maximum pressure of 6 bar was achieved. The pressure was released to atmospheric pressure and 60 g of ESO were added, the temperature was kept for another 5 minutes and then cooled down to ambient temperature (25° C.) within 25 minutes. The yield of sterol release was 98.0%.

### Example 7

0.9 kg of TOP were mixed together with 60 g of ethylenediamine (1.0 moles) obtained from Aldrich Chemical Co., and transferred into a pressure reactor. Within 20 minutes temperature was increased from initially 25° C. to 275° C. and maintained for another 20 minutes, a maximum pressure of 4 bar was achieved. The pressure was released to atmospheric pressure and 60 g of ESO were added, the temperature was kept for another 5 minutes and then cooled down to ambient temperature (25° C.) within 25 minutes. The yield of sterol release was 95.0%.

Step c)—Concentrating the Sterols using Short Path Distillation:

### Example 8

1 kg of the product from example 1 was distilled off in a short path evaporator (SPE) which was operated at 0.1 mm Hg, 290° C. and a feed flow of 600 ml/hour. The residue 1 leaving the bottom of the SPE represented 55% w/w of the CTO feed. The top fraction 2 was representing 45% w/w with 24% of free sterols. The sterols recover yield in this step—amount of concentrated free sterols after distillation compared to amount of free sterols from steps a and b used for distillation—was 83%.

### Example 9

1 kg of the product from example 3 was distilled off in a SPE which was operated at 0.1 mm Hg, 290° C. and a feed flow of 600 ml/hour. The residue 1 leaving the bottom of the WFE represented 67% w/w of the feed. The top fraction 2 was representing 33% w/w with 40% of free sterols. The sterols recover yield in this step was 97.1%.

This sterols have further been purified by a one step crystallization:

80 g of the crude sterols were dissolved in 184 g of n-heptane at 65° C. in a jacketed glass reactor of 1000 ml with a diameter of 105 mm equipped with a KPG-stirrer (plate stirrer) and a reflux cooler. After achieving a clear solution 16 g of methanol/DI-water (50/50 wt %) was added. Crystallization of sterols started immediately. The slurry was further cooled down to a product temperature of 25° C. in 40 minutes. The crystallized slurry was filtered using the Buchner vacuum funnel to filtrate off the mother liquor. The residual cake was washed two times with 100 ml of fresh n-heptane at ambient temperature. The final sterol potency was 98.2%.

### Example 10

1 kg of the product from example 2 was distilled off in a SPE which was operated at 0.1 mm Hg, 290° C. and a feed flow of 600 ml/hour. The residue 1 leaving the bottom of the WFE represented 73.4% w/w of the feed. The top fraction 2 was representing 26.6% w/w with 28.6% of free sterols. The sterols recover yield in this step was 73%.

### Example 11

0.9 kg of the product from example 6 was distilled off in a SPE which was operated at 0.1 mm Hg, 280° C. and a feed flow of 600 ml/hour. The residue 1 leaving the bottom of the WFE represented 67% w/w of the feed. The top fraction 2 was representing 33% w/w with 38% of free sterols. The sterols recover yield in this step was 94.8%.

These sterols have further been purified by a one step crystallization:

80 g of the crude sterols were dissolved in 192 g of n-hexane at 65° C. in a jacketed glass reactor of 1000 ml with a diameter of 105 mm equipped with a KPG-stirrer (plate stirrer) and a reflux cooler. After achieving a clear solution 8 g of methanol/DI-water (50/50 wt %) was added. Crystallization of sterols started immediately. The slurry was further cooled down to a product temperature of 25° C. in 40 minutes. The crystallized slurry was filtered using the Buchner vacuum funnel to filtrate off the mother liquor. The residual cake was washed two times with 100 ml of fresh n-hexane at ambient temperature. The final sterol purity was 98.5%.

1-13. (canceled)

14. A process for the recovery of sterols from sterol and sterol derivative containing organic feed material, which comprises:
a) reacting the organic feed material containing free sterols and sterol derivatives with at least one member selected from the group consisting of polyols, polyamines, alkanolamines and alcohols to convert the sterol derivatives to free sterols, to provide a mixture containing free sterols and residual components;
b) reacting the residual components and their esters or amides with an epoxydated component to provide an epoxylated mixture; and
c) separating the free sterols from the epoxylated mixture by distillation.

15. The process according to claim 14, wherein, the sterol containing organic material comprises at least one member selected from the group consisting of vegetable oil distillates, deodorizer distillates, residues of fatty acid production, residues of fatty acid ester production, soap stock fatty acid residues, sugar cane wax, crude tall oil and tall oil pitch.

16. The process according to claim 14, wherein, the epoxydated components used in step b) comprises at least one member selected from the group consisting of epoxydated soy bean oil, epoxydated linseed oil, epoxydated sunflower oil, epoxydated lard oil, epoxydated fatty acids and epoxydated fatty acid esters.

17. The process according to claim 14, wherein, in step b, the epoxydated component is added in an amount of 2 to 20 wt % based on a weight of organic material feed in step a).

18. The process according to claim 14, wherein, step a) is carried out in the presence of a metal based catalyst.

19. The process according to claim 14, wherein, step a) is carried out without addition of a catalyst.

20. The process according to claim 14, wherein, steps a) and b) are conducted as a batch process or as a continuous process.

21. the process according to claim 14, wherein, transesterification and/or transamidation of step a) is conducted at a temperature of 150° C. to 290° C. in a batch process.

22. The process according to claim 14, wherein, the transesterification and/or transamidation of step a) is conducted at increasing temperatures up to 240° C. to 330° C. in a continuous process.

23. The process according to claim 14, wherein, step b) is conducted at a temperature of 120° C. to 220° C.

24. The process according to claim 14, wherein, the molar ratio of the at least one member in step (a) in relation to a saponification number of the organic feed material is 0.5 to 6.

25. The process according to claim 14, wherein, step c) is conducted by at least one process selected from the group consisting of short path distillation, thin film evaporation, flash evaporation and molecular distillation.

26. The process according to claim 14 further comprising a crystallisation step (d) carried out after step c) for further purification of the separated sterols.

27. The process according to claim 16, wherein, the epoxydated component comprises at least a member selected from the group consisting of epoxydated tall oil fatty acids, oleic acid and linoleic acid and epoxydated tall oil fatty acid esters, oleic acid esters and linoleic acid esters.

28. The process of claim 18, wherein, the catalyst comprises at least one member selected from the group consisting of organotin catalysts, zinc oxide, sodium hydroxide, potassium hydroxide and lithium hydroxide.
